(19) United States
(12) Patent Application Publication
(10) Pub. No.: US 2026/0198900 A1
(43) Pub. Date: Jul. 16, 2026

(54) SYSTEMS AND METHODS FOR IMPROVING ULTRASOUND IMAGE QUALITY

(71) Applicant: Maui Imaging, Inc., Tucson, AZ (US)

(72) Inventors: Elias M. ATMEH, San Jose, CA (US); Bruce R. RITZI, Sunnyvale, CA (US); David J. SPECHT, Tucson, AZ (US)

(21) Appl. No.: 19/315,163

(22) Filed: Aug. 29, 2025

Related U.S. Application Data
(63) Continuation of application No. 18/251,421, filed on Nov. 2, 2021, now Pat. No. 12,514,564.
(60) Provisional application No. 63/108,809, filed on Nov. 2, 2020.

(51) Int. Cl. A61B 8/00; G06T 5/70

(52) U.S. Cl. CPC A61B 8/5269; A61B 8/4477; A61B 8/5207; G06T 5/70; G06T 2207/10136

(57) ABSTRACT

Systems and methods are provided for improving ultrasound image quality. In some embodiments, data sets can be formed from received echoes at each of a plurality of receive elements. The data between the data sets can be masked to include or exclude data. The masked data sets can then be beamformed to form ultrasound images.

### CROSS-REFERENCE TO RELATED APPLICATIONS

This application is a continuation of U.S. patent application Ser. No. 18/251,421, filed May 2, 2023, titled “SYSTEMS AND METHODS FOR IMPROVING ULTRASOUND IMAGE QUALITY,” now U.S. Patent Application Publication No. 2024/0000435, which is the U.S. national phase under 35 U.S.C. § 371 of International Patent Application No. PCT/US2021/057725, filed Nov. 2, 2021, titled “SYSTEMS AND METHODS FOR IMPROVING ULTRASOUND IMAGE QUALITY,” now International Application Publication No. WO 2022/094465, which claims the benefit of U.S. Provisional Patent Application No. 63/108,809, filed Nov. 2, 2020, titled “Systems and Methods for Improving Ultrasound Image Quality,” herein incorporated by reference in its entirety.

This application is also related to the following US Patent Applications: U.S. Pat. No. 8,007,439 titled “Method and Apparatus to Produce Ultrasonic Images Using Multiple Apertures”; U.S. Pat. No. 9,247,926 titled “Multiple Aperture Medical Ultrasound Transducers”; U.S. Pat. No. 9,146,313 titled “Point Source Transmission and Speed-of-Sound Correction Using Multi-Aperture Ultrasound Imaging”; U.S. Pat. No. 9,668,714 titled “Systems and Methods for Improving Ultrasound Image Quality by Applying Weighting Factors”; U.S. Pat. No. 9,986,969 titled “Ultrasound Imaging System Memory Architecture”; U.S. Pat. No. 9,572,549 titled “Calibration of Multiple Aperture Probes”; U.S. Pat. No. 9,510,806 titled “Alignment of Ultrasound Transducer Arrays and Multiple Aperture Probe Assembly”; U.S. Pat. No. 9,883,848 titled “Ultrasound Imaging Using Apparent Point-Source Transmit Transducer”; U.S. Pat. No. 10,401,493 titled “Network-Based Ultrasound Imaging System”; U.S. Pat. No. 10,380,399 titled “Ultrasound Imaging Systems and Methods for Detecting Object Motion”; and US Patent Application Publication 2017/0209121 titled “Ultrasound Imaging with Sparse Arrays”. The entire contents of each of these patents and patent applications is incorporated herein by reference. These related applications may be referred to herein as “Applicant's prior patents and applications.”

### INCORPORATION BY REFERENCE

Unless otherwise specified herein, all patents, publications and patent applications mentioned in this specification are herein incorporated by reference to the same extent as if each individual publication or patent application was specifically and individually indicated to be incorporated by reference.

### FIELD

This invention generally relates to ultrasound imaging and more particularly to systems and methods for improving ultrasound imaging quality by minimizing noise.

### BACKGROUND

In conventional ultrasonic imaging, a focused beam of ultrasound energy is transmitted into body tissues to be examined and the returned echoes are detected and plotted to form an image. While ultrasound has been used extensively for diagnostic purposes, conventional ultrasound has been greatly limited by depth of scanning, speckle noise, poor lateral resolution, obscured tissues and other such problems.

In order to insonify body tissues, an ultrasound beam is typically formed and focused either by a phased array or a shaped transducer. Phased array ultrasound is a commonly used method of steering and focusing a narrow ultrasound beam for forming images in medical ultrasonography. A phased array probe has many small ultrasonic transducer elements, each of which can be pulsed individually. By varying the timing of ultrasound pulses (e.g. by pulsing elements one by one in sequence along a row), a pattern of constructive interference is set up that results in a beam directed at a chosen angle. This is known as beam steering. Such a steered ultrasound beam may then be swept through the tissue or object being examined. Data from multiple beams are then combined to make a visual image showing a slice through the object.

Traditionally, the same transducer or array used for transmitting an ultrasound beam is used to detect the returning echoes. This design configuration lies at the heart of one of the most significant limitations in the use of ultrasonic imaging for medical purposes: poor lateral resolution. Theoretically, the lateral resolution could be improved by increasing the width of the aperture of an ultrasonic probe, but practical problems associated with increased aperture sizes have kept apertures small. Unquestionably, ultrasonic imaging has been very useful even with this limitation, but it could be more effective with better resolution.

Significant improvements have been made in the field of ultrasound imaging with the creation of multiple aperture imaging, examples of which are shown and described in Applicant's prior patents and applications. Multiple aperture imaging methods and systems allow for ultrasound signals to be both transmitted and received via separate apertures.

### SUMMARY OF THE DISCLOSURE

A method of reducing noise in ultrasound imaging is provided, comprising the steps of transmitting a first unfocused ultrasound pulse with a multiple-aperture ultrasound array into a region of interest having one or more types of tissue including bones, receiving echoes from the region of interest with one or more receive elements of the multiple-aperture ultrasound array, forming a digital data set for a plurality of pixels in the region of interest for each of the one or more receive elements, conducting an average of all the digital data sets, applying a control to mask data values from the digital data sets using a predetermined mathematical condition that includes or excludes data, producing a masked data set output, and beamforming the masked data set output to produce one or more ultrasound images.

In some embodiments, the predetermined mathematical condition masks data based on a percentage of the average of each digital data set.

In another embodiment, the predetermined mathematical condition eliminates data that falls outside of a predetermined percentage of the average.

In some embodiments, the masked data set output produces one or more 3D ultrasound images.

In some examples, the predetermined mathematical condition masks data based on a percentage of the average of each digital data set.

In one embodiment, the beamforming is performed in real time, and wherein new data may be used to change or select a new masked data set output.

In some examples, the method further includes adjusting the masked data values.

In one embodiment, the method includes transmitting a second unfocused ultrasound pulse into the region of interest and receiving echoes from the region of interest with one or more receive elements.

In some embodiments, beamforming the masked data set output produces an image of all or a subset of the region of interest.

In one embodiment, the echoes may be received from one or more elements of the same array.

In another embodiment, the first unfocused ultrasound pulse is transmitted with more ultrasound transducers than the second unfocused ultrasound pulse.

In some examples, the second unfocused ultrasound pulse is transmitted with more ultrasound transducers than the first unfocused ultrasound pulse.

A method of reducing noise in ultrasound imaging is provided, comprising the steps of transmitting a first unfocused ultrasound pulse with a multiple-aperture ultrasound array into a region of interest having one or more types of tissue, receiving echoes from the region of interest with one or more receive elements of the multiple-aperture ultrasound array, forming a first digital data set for echoes received by a first receiver element, forming a second digital data set for echoes received by a second receiver element, identifying groups of similar data in the first and second digital data sets, correlating the similar data where common values are shared between the first and second digital data sets, masking data where common values are shared, reducing gap values in the first and second digital data sets to zero or another value, producing a masked data set output, and beamforming the masked data set output to produce one or more ultrasound images.

A method of reducing noise in ultrasound imaging is provided, comprising the steps of transmitting a first unfocused ultrasound pulse with a multiple-aperture ultrasound array into a region of interest having one or more types of tissue, receiving echoes from the region of interest with one or more receive elements of the multiple-aperture ultrasound array, forming a first digital data set for echoes received by a first receiver element, forming a second digital data set for echoes received by a second receiver element, identifying groups of similar data in the first and second digital data sets, correlating the similar data where common values are shared between the first and second digital data sets, masking data where common values are shared, keeping only data retained from gaps between the first and second digital data sets, producing a masked data set output, and beamforming the masked data set output to produce one or more ultrasound images.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A is a schematic illustration of a multiple aperture imaging probe with three transducer arrays and several points to be imaged.

FIG. 1B are illustrations of received waveform coming from elements on each of the three transducers arrays.

FIG. 1C represents digital data information by channel associated with each element of the three transducer arrays.

FIG. 2 is a diagram mapping the process of collecting raw data from a multiple aperture ping based system imaging an region of interest, compartmentalizing data from different elements receiving echo data from the same transmitted ping, identifying large differentials in data, masking out larger values and then beamforming the image.

FIG. 3 is a diagram mapping the process of collecting raw data from a multiple aperture ping based system imaging an region of interest, compartmentalizing data from different elements receiving echo data from the same transmitted ping, averaging data values amongst the data strings, then masking data outside the selectable percentage from the mean, in data, and then beamforming the image.

### DETAILED DESCRIPTION

The various embodiments will be described in detail with reference to the accompanying drawings. References made to particular examples and implementations are for illustrative purposes, and are not intended to limit the scope of the invention or the claims.

The present disclosure provides systems and methods for improving the quality of ultrasound images produced by ping-based multiple aperture imaging (“PMA” imaging) systems by mitigating or eliminating noise in a target image point (i.e., a given pixel or voxel), where such noise is caused by a temporally-neighboring structure with a very different echogenic character than the target image point. Such noise will be referred to herein as “neighbor noise.”

As described in more detail below and in Applicant's prior patents and applications, PMA imaging involves transmission of ultrasound “pings,” echoes of which may be received by receive elements located at some distance from the transmitter. Each received echo signal lies along an ellipse defined by the transmitter and receiver positions and the time interval between ping transmission and echo reception. An image may be formed by combining such ellipses (i.e., data samples) in a way that their intersections become emphasized. Each intersection may be an image point in a two-dimensional or three-dimensional image. However, if one erroneous ellipse has a substantially higher or lower value than others contributing to the same image point, the erroneous ellipse's value may dominate and cause the image point to be brighter or darker than the real object represented by the image point.

An erroneous ellipse may contribute to an image point when both a bright object and a dark object lie on the same temporal ellipse (i.e., “neighboring ellipses”). This may occur due to differences in materials or different structures along paths between transmitter, reflector object, and receiver, or due to brighter and darker reflectors being located near one another within an imaged region of interest. For example, this phenomenon may become problematic when imaging a spinal cord immediately inside the epidural space and surrounded by strong reflectors of the vertebral bones. Similarly, when imaging inside a skull using PMA systems, a (relatively dim) ventricle of the brain may not be distinguishable from the (relatively bright) skull when the ventricle is located immediately next to the interior surface of the skull. In these and many other imaging scenarios, a summation for a given image point representative of a given reflector may inadvertently include one or more ellipses representative of a neighboring structure with a substantially different echogenic character (i.e., substantially brighter or darker) than the given reflector. This “neighbor noise” phenomenon can have the effect of blurring edges and obscuring real structures that might otherwise be visible in the image.

In some embodiments, neighbor noise may be mitigated or eliminated by evaluating data samples contributing to an image or an image region to identify data samples containing an unacceptable degree of neighbor noise. Once identified, the impact of such neighbor noise data samples on a final image may be minimized. In some embodiments, sub-visual data analysis may be used to identify data samples, groups of data samples, or image point regions likely to contain data samples with substantial neighbor noise.

Noisy data samples may generally be identified by mapping all collected data samples to respective image points (e.g., pixels or voxels), then for each image point, collectively evaluating the set of data samples contributing to the image point and identifying individual data samples containing unacceptably high levels of noise relative to the set. Once identified, the impact of noisy data samples on a final image may be mitigated or eliminated by adjusting, weighting, ignoring, or otherwise modifying the value of the noisy data sample. Various examples and embodiments of such methods are described herein.

Although the various embodiments are described herein with reference to ultrasound imaging of various anatomic structures or implanted medical devices, it will be understood that many of the methods and devices shown and described herein may also be used in other applications, such as imaging and evaluating non-anatomic structures and objects.

### Ping-Based Multiple Aperture Imaging

Some embodiments of the systems and methods described herein are based on a unique imaging modality referred to as ping-based multiple aperture imaging (“PMA” imaging). An introductory description of ping-based multiple-aperture imaging is provided below. Additional details, examples, embodiments, and applications of methods and structures useful in performing ping-based multiple aperture imaging are described in Applicant's prior patent applications referenced above.

Briefly, PMA imaging involves transmitting a series of un-focused two-dimensional or three-dimensional “pings” into a medium from a “transmit aperture” (which may be made up of one transducing structure or a group of transducers operating in concert) then receiving and storing signals produced by echoes and/or through-transmissions of each ping. Signals are received by many “receive elements” (each made up of one or more transducing structures) which may be grouped into “apertures.” The receiving transducers produce time-varying analog signals with amplitudes proportional to an intensity of energy impinging on the transducer. Such analog signals may be digitally sampled at a sampling rate, and digital samples may then be stored. The value of each digital sample may be proportional to the intensity of received ultrasound. Each digital sample may represent an “echo” of some reflective or transmissive structure in the medium. Digital samples received by a single receive transducer element may be organized in “strings” of data samples, which may be sub-divided into “sub-strings” as described in some embodiments herein. An image may be formed by mapping the samples to locations within the imaged medium and assigning brightness (and/or color) values to each image point (e.g., a pixel or voxel) in proportion to the value of contributing data samples.

While terms such as “bright” and “dark” are used herein to refer to image points and data samples, the skilled artisan will recognize that such terms are not absolute, as the brightness or contrast of a displayed image may be adjusted. Instead, the terms are used in a relative sense to distinguish those data samples and image points representative of highly reflective or “echogenic” structures which are typically but not necessarily referred to as being more “bright” than minimally-reflective structures which are typically but not necessarily referred to as “dark.” Of course, some imaging systems may be configured with an opposite convention in which samples with greater energy intensity are displayed as dark points while samples with less energy intensity are displayed as brighter points. In either convention, in the context of the systems and methods described herein, the term “bright” is intended to refer to points representing points of greater received energy intensity (regardless of whether the energy is received after reflection from or transmission through an imaged structure), while “darker” points are those with relatively lower received energy intensity.

### Beamforming Images in from Ping Based Multiple Aperture Imaging Systems

A complete sub-image of the medium may be obtained from signals produced by each receive element. Sub-images obtained from elements of a common aperture may be combined with one another to produce a “first-level” image. Sub-images and/or first-level images from multiple ping-transmissions (transmitted from the same or different transmit apertures) may be combined to produce “second-level” images. Second-level images from multiple receive apertures may be combined to produce “third-level” images. Many permutations of image-layer combination sequences are possible and therefore sub, first, second, and third level images need not necessarily be formed in the sequence implied by the names.

If the transmit elements and/or the receive elements are spaced from one another in two or three dimensions, the “images” (including sub-images) may be three-dimensional volumes made up of three-dimensional voxels. Any two-dimensional section of such a volume may be selected and displayed as a matrix of two-dimensional pixels. The term “image point” will be used to refer to discrete elements (e.g., pixels or voxels) of a two-dimensional or three-dimensional image.

As signals are received by a transducer element, the signals may be converted into a sequence of digital data, which may be stored in a volatile and/or non-volatile memory device. Each entry in such a sequence of data entries may be referred to as a “data sample”. The term “data sample” may also refer to values obtained by aggregating multiple data entries (e.g., averaging, taking a minimum or a maximum, etc.) or values obtained by interpolating between two or more data entries.

In order to form a sub-image from a collection data samples, each sample (individually, aggregated, or interpolated) must be mapped to its possible location within the image through a process referred to herein as “beamforming.” Each data sample represents a range of potential locations (a locus) within the image determined by the location of the transmit element and receive element, the difference in time between ping transmission and signal reception, and the speed-of-sound through the imaged medium.

In a multiple aperture imaging system in which the transmitter is located at a different point than the receiver, the locus of possible locations for each sample takes the shape of a two-dimensional ellipse or a three-dimensional ellipsoid with the transmit element and the receive element located at the foci of the ellipse or ellipsoid reference U.S. Pat. No. 9,146,313 titled “Point Source Transmission and Speed-of-Sound Correction Using Multiple-Aperture Ultrasound Imaging. The term “locus” (and its plural “loci”) will be used to refer to either an ellipse or an ellipsoid. The imaging system converges on the correct location of each image point by adding together multiple data samples with loci intersecting the same image point. Each data sample contributing to a single image point may be referred to as a “contributor” to that image point. The point at which the ellipses or ellipsoids intersect is reinforced (i.e. has a greater total brightness than its individual contributors) and represents the correct location of the point to be displayed or recorded.

This process is susceptible to a unique form of error referred to herein as neighbor noise. If a particular data sample contains a high degree of noise causing its locus to be substantially brighter than other contributors to an image point, a larger region of the neighbor noise sample may be displayed, creating a noise artifact in the shape of the locus. Such individual neighbor noise samples may create significant distortions of an image by highlighting regions that do not correspond to physical structure in the imaged medium. Distortions caused by neighbor noise may be identified through any of a number of techniques, some of which are described below. Once identified, neighbor noise can be minimized when forming an image by one or more of the techniques described herein.

### Identifying Neighbor Noise Data Samples by Averaging

Highly echogenic reflectors that are substantially brighter than other contributors to the same image points are a problem that can cause neighbor noise. Here a “too bright” contributor that is overwhelming other contributors may create bright artifacts or other false information. This is particularly problematic for image points that would otherwise be relatively “dark” but-for the strong echogenic reflector located in the data samples. In an opposite but related fashion, less-echogenic reflectors can be erroneously displayed as much darker than expected because other contributors to the same image point will tend to cancel the effects of a “too dark” contributor. In both cases therefore, it may be beneficial to identify data samples (or ellipses) representing neighbor noise.

In general, for a single image point, data samples resulting from different combinations of transmitted ping and receive element may reveal brighter or darker echoes of a reflector due to differences in path length, look angle, obstacles, materials, time of ping transmission, or other factors. Nonetheless, under normal conditions, the degree of such variations can be expected to remain within predictable ranges which may be determined based on empirical testing and/or mathematical modeling/simulation. Echo values that fall significantly outside of such expected ranges are likely to be noise or other forms of error. Therefore, it may be desirable to systematically define “abnormally bright” values, identify data samples contributing “abnormally bright” values to any image point, and to minimize the deleterious impact of such abnormally bright samples.

In some embodiments, instead of evaluating every image point within the medium for high noise, the set of image points to be evaluated may be reduced to a candidate set of image points. For example, in some embodiments image points with brightness values less than a pre-determined value (e.g., ≤0.9 on a scale of 0.0 to 1.0) may be selected for analysis to detect neighbor noise contributors. In other embodiments, image points with brightness values greater than a predetermined lower value (e.g., 0.1 on a scale of 0.0 to 1.0) but less than a pre-determined upper value (e.g., 0.8) may be selected for analysis to detect neighbor noise contributors.

In some embodiments, image points to be evaluated for the existence of neighbor noise data samples may be identified based on an analysis of adjacent image points, or image points within a region. For example, if after applying all contributors, a particular image point has a brightness value substantially higher than all adjacent image points or all image points within a region, that image point may be selected for evaluation of contributors as possible neighbor noise contributors. In other embodiments, an image point to be evaluated for the existence of neighbor noise data samples may be identified by evaluating data samples contributing to a group of image points in a region so as to detect an “edge” between relatively darker and lighter image points in the region. An example of such a process is described in the section below.

Whether evaluating all image points or a sub-set of image points selected by a method such as those described above, various processes may be used for identifying neighbor noise contributors to a particular image point. In one example embodiment, such a process may comprise transmitting a ping from a transmit aperture, receiving reflected and/or transmitted signals from the ping, digitizing and storing sampled digital data representing the received signals, and beamforming the stored data to map data samples to image points. Then for each image point to be evaluated:
determining an aggregate value of the set of data samples contributing to the image point, and identifying neighbor noise contributors as those data samples with values varying from the aggregate value by greater than an expected variance.

In various embodiments, the step of evaluating data samples to identify neighbor noise contributors may be performed before and/or after various coherent or incoherent summation steps as described in the various applications referenced above and incorporated herein by reference. For example, in some embodiments, raw data samples may be evaluated before any data summation steps in order to detect edge regions or other distinguishable features with a much greater degree of detail than may be possible after data summation.

In various embodiments, the “aggregate value” of a set of data samples contributing to a particular image point may be the arithmetic mean (simple average), median (the midpoint of all values of samples in the set), the mode (the most frequent value in the set), the maximum (the largest value of the set), the minimum (the smallest value of the set) or other value describing or obtained from the set of data samples.

In various embodiments, the variance from an aggregate value defining a neighbor noise data sample may be defined in numerous ways. For example, the variance may be a fixed numerical value, a multiple of the aggregate value, a percent change from the aggregate value, a number of standard deviations above the aggregate value, a percentile of the set of data samples contributing to the image point, or other metrics of variance from the aggregate value.

In some embodiments, neighbor noise contributors to an image point may be defined as samples with brightness values at least N times greater than the mean, median, mode, maximum, or other aggregate value of the set of contributors to the image point. In such embodiments, N may be at least about 1.0 up to about 2.0 or more.

In other embodiments, neighbor noise contributors may be defined as samples with brightness values more than N standard deviations greater than the mean value of contributors to the image point. In other embodiments, neighbor noise contributors may be defined as samples with brightness values greater than the maximum value of the set of contributors, or N times the maximum, or more than M% greater than the maximum. In other embodiments, neighbor noise contributors may be defined as samples with brightness values greater than the N times the mode, where the “mode” is defined as the most frequently occurring value in the set of data samples contributing to the image point. In some embodiments, the mode may be determined based on rounded values of the data samples (e.g., by rounding each value of the set to a predetermined number of digits and then determining the most frequent value).

In some embodiments, mathematical or other evaluations of the raw data samples collected by multiple transducer elements from a PMA system may be done before image beamforming in order to identify data samples to be adjusted. In some embodiments, such pre-beamforming evaluation may be used for other analyses such as object recognition or others.

### Identifying Neighbor Noise Data Samples by Edge Detection

A limitation of Ping Based Multiple Aperture Imaging (PMA) systems occurs when a hard tissue reflector or group of reflectors is located immediately adjacent to a soft tissue reflector or group of reflectors. In such instances, echo data samples contributing to a pixel or voxel that represents a hard tissue or highly echogenic reflector (i.e., a “bright” reflector image point) may be assigned a significantly stronger value on a grey scale than echo data samples representing a soft tissue or less-echogenic reflector (i.e., a relatively “dark” image point) immediately adjacent to the hard reflector. This noise may be created by both stronger and weaker echo signals being located on the same temporal ellipse relative to an ultrasound receiving element on a multiple aperture transducer array. Therefore, the ability to more accurately detect edges between hard (bright) reflectors and relatively softer (darker) reflectors may be used to enhance those edges in a final displayed image. Such “edge detection” techniques may also be useful in enabling automated analysis of raw echo data to produce actionable information even before producing an image.

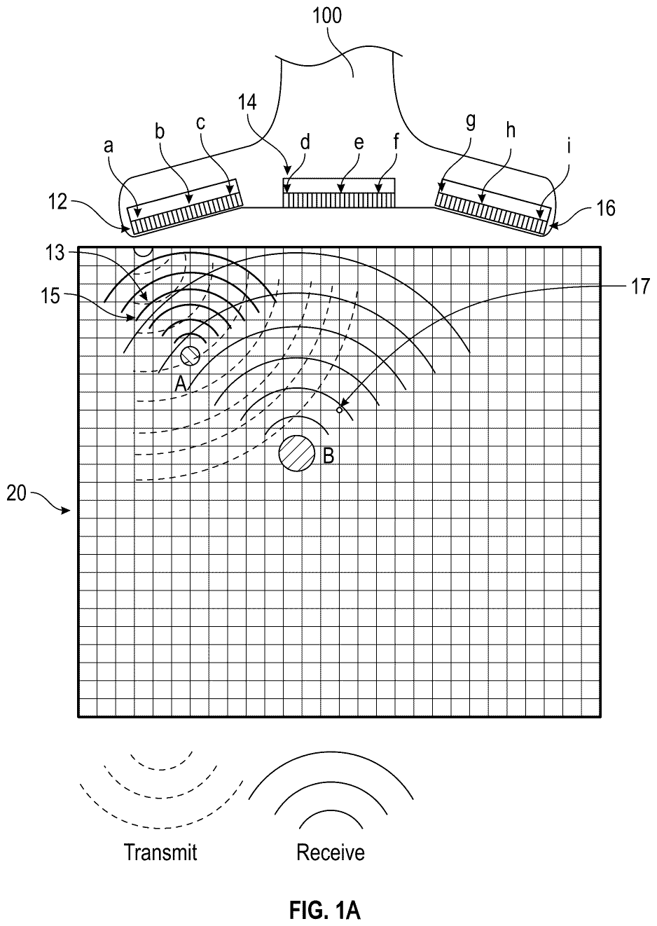

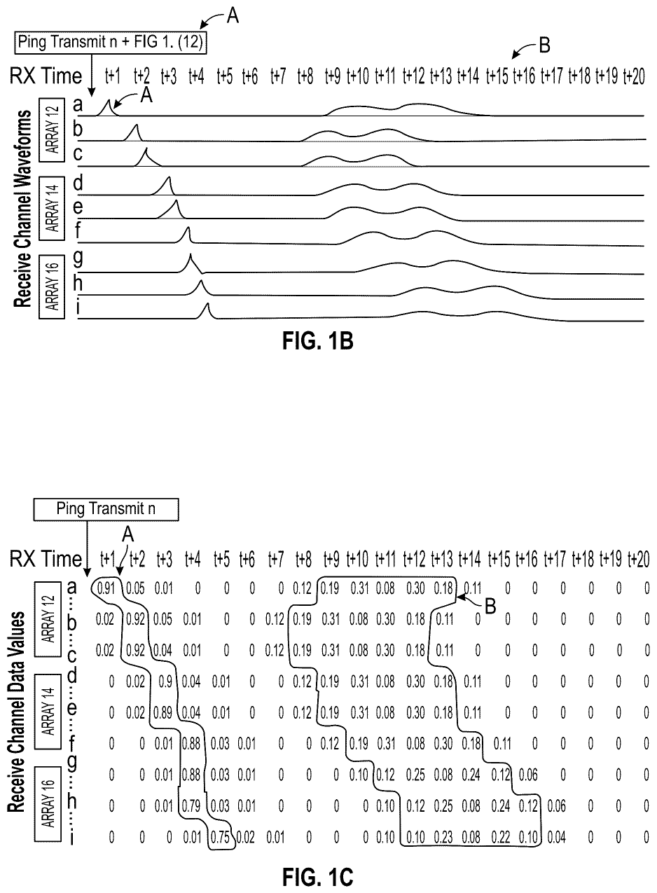

FIG. 1A demonstrates a ping based multiple aperture probe 100 against a skin surface S, with arrays 12, 14, and 16. Subarrays or often individual elements within each array are indicated as points a, b, c, d, e, f, g, h and i. However, sub-arrays can be located across physical gaps between arrays and should not be considered limited to individual elements on an individual array. A ping transmission is represented by the wavefront 13 (dashed wavefront(s)) generated by a transmit aperture at ‘a’ on array 12 and is indicated by wavelets. Point A in the medium or tissue 20 is meant to represent a hard structure (e.g., calcium or hardened plaque from atherosclerosis, or other hard objects such as bone), which would immediately reflect or scatter the transmitted wavefront 13 in multiple directions represented here as reflected wavefront 15 (solid wavefront(s)). The reflected wavefront emanating from point A may provide a relatively bright signal to the receive elements in arrays 12, 14, and 16. The transmitted wavefront 13 may also continue on through the medium or tissue 20 to point B, which is meant to represent an anechoic structure (e.g., a blood vessel or other soft tissue) that would provide a relatively weaker reflected wavefront 17 back to the elements on arrays 12, 14, and 16.

FIG. 1B is an illustration of waveforms representing the amplitude and timing of wavefronts 13 and 17 respectively returning to receivers a, b, c, d, e, f, g, h and i on each of the three arrays 12, 14, and 16 of FIG. 1A. As shown in FIG. 1B, the amplitude of reflections from objects A and B can be seen. Note that the amplitude of object at B is stronger at the edges of the object and weakens inside the mostly anechoic object interior. The reflections are received at different times on the array based on the distance from each receive element to the object. For example, it can be seen that element a on array 12 is closer to object A, as the reflection is received at time t+1. In contrast, element I on array 16, which is furthest from object A, does not receive the reflection until time t+4. The same can be seen for object B, with elements b, c, and d being the closest and element i being the furthest from object B.

FIG. 1C is a table indicating digital data sample values associated with signal strength being stored from each receiver's data channel relative to a ping's transmission. The circumscribed data samples in FIG. 1C suggest that a strong reflector A is located near Array 12 element a, and the amplitude of that signal can be mapped through as it is received on elements across the probe. As shown, this data represents Point A in the medium 20; however, even without beamforming and even as reflector strength drops off the further the echo is being received from the origin or the reflector, it is still clear that this point can be mapped and tracked while still in the digital realm. In other words, the point A may be detected and distinguished by analysis of the data samples even without beamforming and displaying an image.

Further in FIG. 1C, as time progresses on Array 12 element a toward t+10 a small signal builds, then is followed by almost no signal at t+11, then a small signal again in t+12 followed by almost no signal. This data would represent Point B if an image were to be beamformed from it. However, that same pattern of small signals bracketing a weak signal appear in the channels going down through the probe data even though being received at slightly different times. The pattern appears to be closest to Array 12 elements b and c, then gets further away when moving onto Arrays 14 and 16.

Regardless of the individual ping transmission observed in FIGS. 1B and 1C, current Ping Based Multiple Aperture systems at this point compound the total data from a selectable sequences of pings (e.g. 13, 15 . . . n) and associated reflected data un-beamformed raw data from all receivers to create a single data set. This data set then is beamformed into a pixelated image. This compounded beamforming can subsequently enable the noise to be accrued from a strong target being adjacent to a weak target.

### Mitigating Neighbor Noise Data Samples

Once identified, the impact of neighbor noise contributors on an image point may be mitigated in various ways. In some embodiments, data samples identified as containing bright high noise may be weighted by a pre-determined fractional adjustment factor. For example, the brightness of a neighbor noise data sample may be multiplied by an adjustment factor of between about 0.5 and 0.001 or smaller. Example adjustment factors may include 0.50, 0.45, 0.40, 0.35, 0.30, 0.25, 0.20, 0.15, 0.10, 0.05, 0.04, 0.03, 0.02, 0.01, 0.005, 0.001, or smaller weights. In other embodiments, a neighbor noise data sample may be re-set to be equal to an aggregate value of its co-contributors. For example, a neighbor noise data sample may be re-set to be equal to a median, mean, mode, minimum, or other value obtained from analysis of the set of contributors that includes the identified neighbor noise sample. In other examples, a neighbor noise data sample may be re-set to equal to or less than a pre-determined number of standard deviations from a mean, median, mode or other aggregate value. In still other embodiments, a neighbor noise data sample may be re-set to a brightness value of zero or a pre-determined minimal value. In some embodiments, a set of adjustment factors for an entire image or a region of an image may be referred to herein as a “mask” applied to the image or region. Such a mask may simply be an array of adjustment factors corresponding to image points and/or data samples used to form the image or image region.

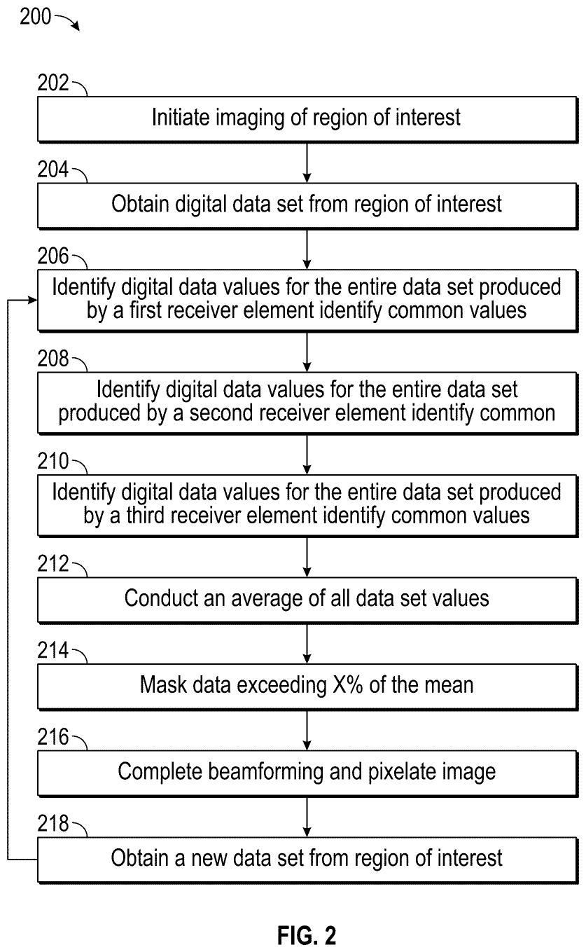

In one embodiment, neighbor noise can be substantially reduced by use of a data masking technique such as the Process 200 illustrated in FIG. 2. Step 202 indicates that PMA systems are used in their standard data collection format by the user to image the region of interest. This can include transmitting one or more unfocused ultrasound pulses from the PMA system into a region of interest. In some embodiments, the region of interest can include one or more types of tissue including bones. While PMA systems may utilize beamforming in real time to present an image to the user, the PMA system may also collect data sets continuously. In fact, the image that is presented during the imaging session may simply be a subset of the data that is available to be beamformed into an image and may be purely selectable by the user either in real-time on the system or remotely via cloud operation. Step 204, therefore, highlights the fact that the PMA system is collecting a data set from the entire region of interest, not just the image that is being presented. This can include receiving echoes from tissue and converting the echoes to a digital channel data on one or more channels.

FIG. 2, step 206 then begins the process of analyzing the data in the region of interest. Data being collected after Analog to Digital conversion is stored into data strings for a first Receiver Element. This receiver element may be part of an array, or it may be an independent element used as an omni-directional receiver. It need not be used in conjunction with other elements to collect and compound data in real time. Step 208 therefore, highlights that a second receiver element is used to produce a second string of data coming off of the same ping utilized to provide receiver element data to the receiver in step 206. Similarly, echo data coming off of the same ping transmission for 206 and 208 is used by a third receiver element described in step 210.

A processor is then utilized in step 212 to conduct an average of all data set values for all data strings. In some implementations, the data string may be collected for an entire region of interest (i.e. large sample period). In other implementations, the data may be collected for only a specific pixel (i.e. specific sample period). The user may then select a control to mask data based on a percentage of the mean data for each string as described in step 214. The mask eliminates data, both high and low, that falls outside of the percentage of mean range. Conversely, the mask could be utilized to eliminate data inside the percentage of the mean range, and thus keep the “outliers.” Step 216 utilizes the remaining data in the beamforming process to create pixelated images. In the case of 3D imaging, the same process can be utilized to create voxel images. Step 218 indicates that the process can be repeated. When imaging in real time, new data may be used to change or select a new mask value. When dealing with a stored or static data set, the adjustment mask values can be adjusted on the existing data set.

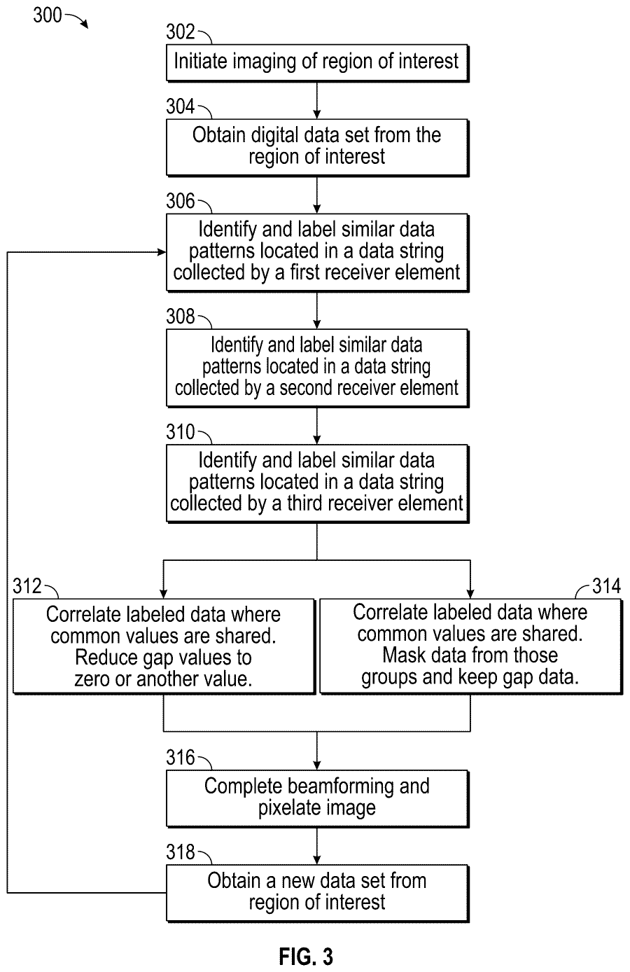

In one embodiment, neighbor noise can be substantially reduced by grouping digital data as outlined on FIG. 3, Process 300. Step 302 indicates that PMA systems are used in their standard data collection format by the user to image the region of interest. Step 304, highlights the fact that the PMA system is collecting a data set from the entire region of interest, not just the image that is being presented. Step 306 uses artificial intelligence to group data of similar values on different data strings into known object locations. This is best illustrated in FIG. 1A and FIG. 1B. FIG. 1B represents a digital data string for elements a through i along different arrays 12, 14 and 16 over time periods t+1 through t+20. Although appearing at different times in different data strings, a correlation of the existence of a strong reflector (A) is noticeably present in each data string. The presence, when compared to surrounding reflectors, is differentiated even though weaker discernment is apparent as the echo reaches receiver elements that are further away from point A in FIG. 1A. Similarly, the appearance of moderate signal strength, then weaker signal, then stronger again in a saddle type shape can be discerned in the data strings presumably representing point B in FIG. 1A. Although the amplitude of this reflector is weaker and dynamic, there is a definite correlation that can be made within receiver data strings even though the time of receipt differs.

Step 306 of FIG. 3 uses data correlations or artificial intelligence to designate similar data patterns as groups or fiducials that will ultimately be displayed together in an image. While in the data string state, they become named or numbered groupings or fiducials. Steps 308 and 310 show this same process being conducted for elements for a second and third receiver elements. Step 312 empowers the system to either automatically or manually reduce the gaps between labeled or fiducial groupings to zero. Conversely, Step 314 empowers the system to mask out labeled or fiducial groups and keep only the data retained from the gaps in between fiducial groups. A control in the system can be configured to alternate between methods 312 and 314 automatically or enable the user to select between Step 312 or Step 314.

Step 316 utilizes the now filtered data to be beamformed into an image. Step 318 begins the process of obtaining new data. Image data can also be stored after step 316, saved in memory, and then added to data using the other filter path.

Any of the foregoing embodiments may be used in combination with a multiple aperture imaging probe of any desired construction. Examples of multiple aperture ultrasound imaging probes are provided in Applicant's prior patent applications referenced herein.

Embodiments of the systems and methods described above may also be beneficially applied to multiple aperture ultrasound imaging systems utilizing focused phased array transmit pulses rather than point source transmit pulses (pings). Similarly, embodiments of the systems and methods described above may also be beneficially applied to single-aperture imaging systems using multiple sub-apertures for ping transmission. In still further embodiments, the methods described above may also be applied to conventional ultrasound systems using phased array-transmissions from a single-aperture probe.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. Various modifications to the above embodiments will be readily apparent to those skilled in the art, and the generic principles defined herein may be applied to other embodiments without departing from the spirit or scope of the invention. Thus, it is intended that the scope of the present invention herein disclosed should not be limited by the particular disclosed embodiments described above, but should be determined only by a fair reading of the claims that follow.

In particular, materials and manufacturing techniques may be employed as within the level of those with skill in the relevant art. Furthermore, reference to a singular item, includes the possibility that there are plural of the same items present. More specifically, as used herein and in the appended claims, the singular forms “a,” “and,” “said,” and “the” include plural referents unless the context clearly dictates otherwise. As used herein, unless explicitly stated otherwise, the term “or” is inclusive of all presented alternatives, and means essentially the same as the commonly used phrase “and/or.” It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs.

1. (canceled)

2. A method of evaluating tissue with ultrasound, comprising the steps of:
transmitting a first unfocused ultrasound pulse with a multiple-aperture ultrasound array into a region of interest having one or more types of tissue including bones;
receiving echoes from the region of interest with a plurality of receive elements of the multiple-aperture ultrasound array over a time period following transmission of the first unfocused ultrasound pulse;
forming a digital data set for a plurality of pixels in the region of interest, the digital data set comprising a plurality of data strings that include echo amplitudes for each of the plurality of receive elements over the time period;
grouping data patterns of similar values over the time period across different data strings into two or more known object locations; and
classifying a tissue or structure type of at least one of the two or more of the known object locations based on the data patterns of similar values.

3. The method of claim 2, wherein the classifying step comprises classifying a first known object location as comprising a hard tissue structure.

4. The method of claim 3, wherein the hard tissue structure comprises bone.

5. The method of claim 3, wherein the hard tissue structure comprises calcium or hardened plaque.

6. The method of claim 3, wherein the data patterns correlated to the first known object location are represented as a bright signal in the data strings with a sharp peak in the echo amplitudes.

7. The method of claim 2, wherein the classifying step comprises classifying a first known object location as comprising an anechoic structure.

8. The method of claim 7, wherein the anechoic structure comprises a vessel.

9. The method of claim 7, wherein the data patterns correlated to the first known object location are represented as an increased echo amplitude followed by a lower echo amplitude followed by an increased echo amplitude.

10. The method of claim 7, wherein the data patterns correlated to the first known object location are represented as a saddle-shaped waveform.

11. The method of claim 2, further comprising:
conducting an average of all the digital data sets;
applying a control to mask data values from the digital data sets to include or exclude data;
producing a masked data set output; and
beamforming the masked data set output to produce one or more ultrasound images.

12. The method of claim 11, wherein the grouping and classifying steps are performed prior to the beamforming step.

13. The method of claim 11, wherein the control mask is based on a user-selected percentage of the average.

14. The method of claim 2, wherein the control mask eliminates data that falls outside of the user-selected percentage of the average.

15. The method of claim 2, wherein beamforming the masked data set output produces one or more 3D ultrasound images.

16. The method of claim 2, wherein the beamforming is performed in real time, and wherein new data may be used to change or select a new masked data set output.

17. The method of claim 2, further comprising adjusting the masked data values

18. The method of claim 2, wherein beamforming the masked data set output produces an image of all or a subset of the region of interest.

19. The method of claim 2, wherein the echoes may be received from one or more elements of the same array.
